(19) United States
(12) Patent Application Publication
(10) Pub. No.: US 2017/0000198 A1
(43) Pub. Date: Jan. 5, 2017

(54) DEGRADABLE EMERGENCY DIAPER LINER

(71) Applicants: Phuong Thanh Tran, Etobicoke (CA); Curtis Patrick George Messam, Etobicoke (CA)

(72) Inventors: Phuong Thanh Tran, Etobicoke (CA); Curtis Patrick George Messam, Etobicoke (CA)

(21) Appl. No.: 14/789,132

(22) Filed: Jul. 1, 2015

(51) Int. Cl. A41D 13/02; A61M 35/00

(52) U.S. Cl. CPC A41D 13/02; A61M 35/00; A41D 2400/60; A41D 2500/50

(57) ABSTRACT

A degradable emergency diaper liner comprising: an inner and an outer portion; a front portion; a back portion; a bottom portion comprising leg portions; and an open neck portion comprising shoulder straps; wherein the front portion extending from a terminal end of the leg portions and the open neck portion and the back portion extending between a terminal end of the leg portions and the open neck portion, wherein the leg portions comprise leg apertures configured for receiving legs of a wearer, and wherein each of the shoulder straps are coupled to the front portion and back portion adjacent to the open neck portion for receiving a head and arms of a wearer.

### FIELD OF THE INVENTION

The present invention is in the field of a degradable emergency diaper liner and more specifically to a biodegradable and/or compostable emergency diaper liner that is environmentally safe.

### BACKGROUND OF THE DISCLOSURE

Diapers are generally worn by infants who are not potty trained and some adults that may be incapable of controlling their bladder or bowel movements. Diapers come in various sizes and have been made of disposable materials. Disposable diapers are more convenient and aid in the clean up. Disposable diapers in some cases are derived from polymer materials, tape, and an elastic waistband to secure the diaper and its contents.

However, children, infants and even adults are at times in a situation whereby an accidental urination and/or bowl movement occurs. In particular cases the wearer may or may not be wearing a diaper. In the situation where there is no diaper being worn there will be the problem of urine and/or feces leaking through the clothing. In cases where the accident occurs away from the home or elder care facility, hospital, etc. an emergency situation exists, whereby the leakage must be contained. This type of accident could potentially damage an automobile's interior, furniture or otherwise.

Moreover, disposable soiled diapers are merely tossed in the garbage, whereby the diapers are then transported to the landfill and disposed of without any treatment. The biodegradation of human waste is significantly impaired when they are wrapped or enclosed in non-biodegradable plastic films and/or fabrics.

In order to solve the above mentioned problems, there is a need for a degradable emergency diaper liner to be worn over a wearer's clothing in the event of accidental urination and/or bowl movements.

### SUMMARY OF THE INVENTION

A degradable emergency diaper liner comprising A degradable emergency diaper liner comprising: an inner and an outer portion; a front portion; a back portion; a bottom portion comprising leg portions; and an open neck portion comprising shoulder straps; wherein the front portion extending from a terminal end of the leg portions and the open neck portion and the back portion extending between a terminal end of the leg portions and the open neck portion, wherein the leg portions comprise leg apertures configured for receiving legs of a wearer, and wherein each of the shoulder straps are coupled to the front portion and back portion adjacent to the open neck portion for receiving a head and arms of a wearer.

### BRIEF DESCRIPTION OF THE DRAWINGS

FIG. 1 is an illustration of the front portion of the degradable and/or compostable emergency diaper liner.

FIG. 2 is an illustration of the back portion of the degradable emergency diaper liner.

FIGS. 3A, 3B and 3C are illustrations of the back portion fastener 41, 51 and 61, respectively.

### DETAILED DESCRIPTION OF THE EMBODIMENTS

The following is a detailed description of certain specific embodiments of the degradable emergency diaper liner disclosed herein. In this description reference is made to the drawings.

### Definitions

The term “polyolefin” as used herein includes any polymer or resin where an olefin such as polyethylene, polypropylene, etc. is the major component. Furthermore, the term “polyolefin” may include a mixture of polyolefins. Further still, the term “polyolefin” may refer to a primary polyolefin, secondary polyolefin, or combinations thereof.

The term “prodegradant” refers to ingredients that impart the degradable characteristics.

The term “degradable” relates to the polyolefin film which exhibits more than a loss of physical properties but a loss of molecular weight. The degradation of the polyolefin resins is clearly characterized as chemical since the degradation does not require a UV light or a bio-initiator for the degradation to take place. However, UV light can accelerate chemical degradation.

The term “biodegradable” refers to the chemical dissolution of materials by bacteria, fungi or other biological means.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained. It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items. As used herein, the term “comprising” means including elements or steps that are identified following that term, but any such elements or steps are not exhaustive, and an embodiment can include other elements or steps.

### Introduction

In one aspect, disclosed herein are liquid impermeable degradable emergency diaper liner comprising A degradable emergency diaper liner comprising: an inner and an outer portion; a front portion; a back portion; a bottom portion comprising leg portions; and an open neck portion comprising shoulder straps; wherein the front portion extending from a terminal end of the leg portions and the open neck portion and the back portion extending between a terminal end of the leg portions and the open neck portion, wherein the leg portions comprise leg apertures configured for receiving legs of a wearer, and wherein each of the shoulder straps are coupled to the front portion and back portion adjacent to the open neck portion for receiving a head and arms of a wearer, and wherein the liner is sized to fit over the wearer's clothing in the event of a bowl movement and/or urination.

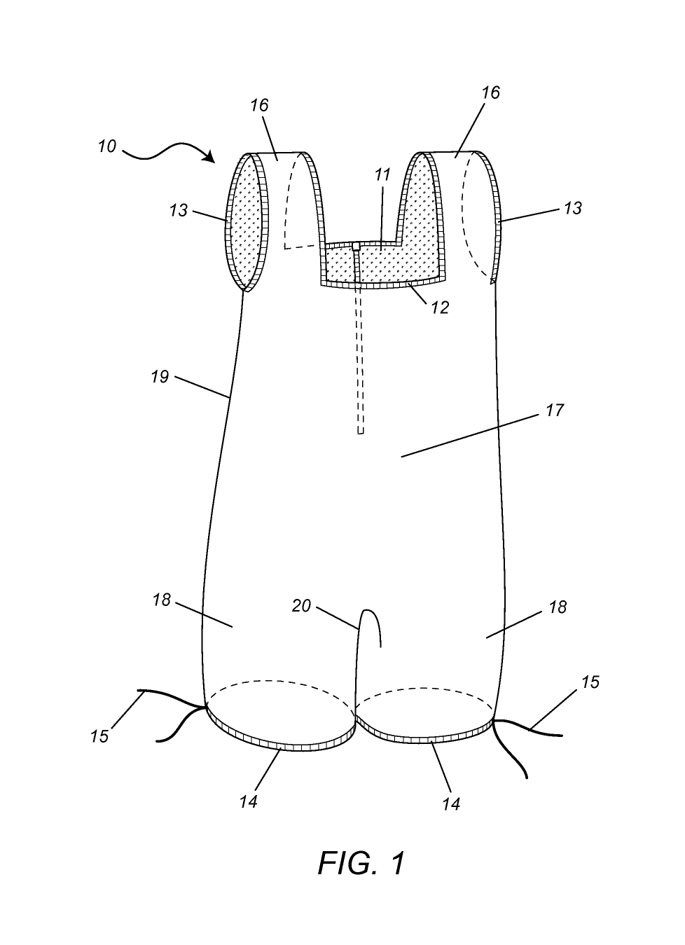

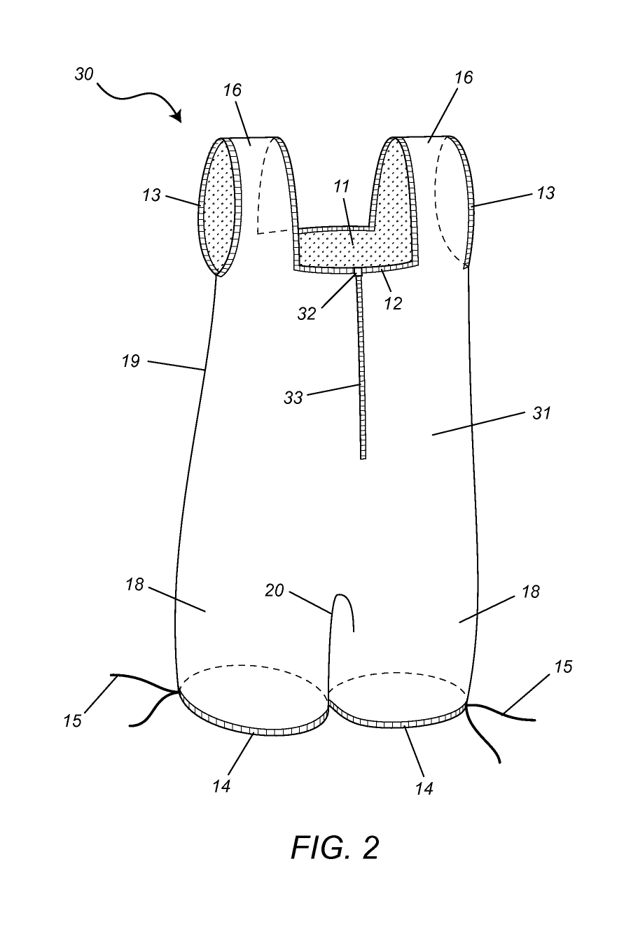

Referring initially to FIGS. 1 and 2, according the present disclosure, a degradable emergency diaper liner front side 10 and back side 30 includes at least one degradable diaper liner front sheet 17 and back sheet 31, respectively. The degradable emergency diaper liner is made of a formulation which includes degradation properties and preferably includes a biodegradable and/or compostable plastic formulation.

Referring to FIGS. 1-3, degradable emergency diaper liner may comprise shoulder portions 16, which hold the neck portion 12 between the waist and the neckline of the wearer. In some embodiments, the neck portion 12 is adjacent to the neckline of the wearer. In some embodiments, the neck portion 12 comprises a hem having a drawstring disposed therein. In some embodiments, the terminal ends of the shoulder portions comprise a hem 13.

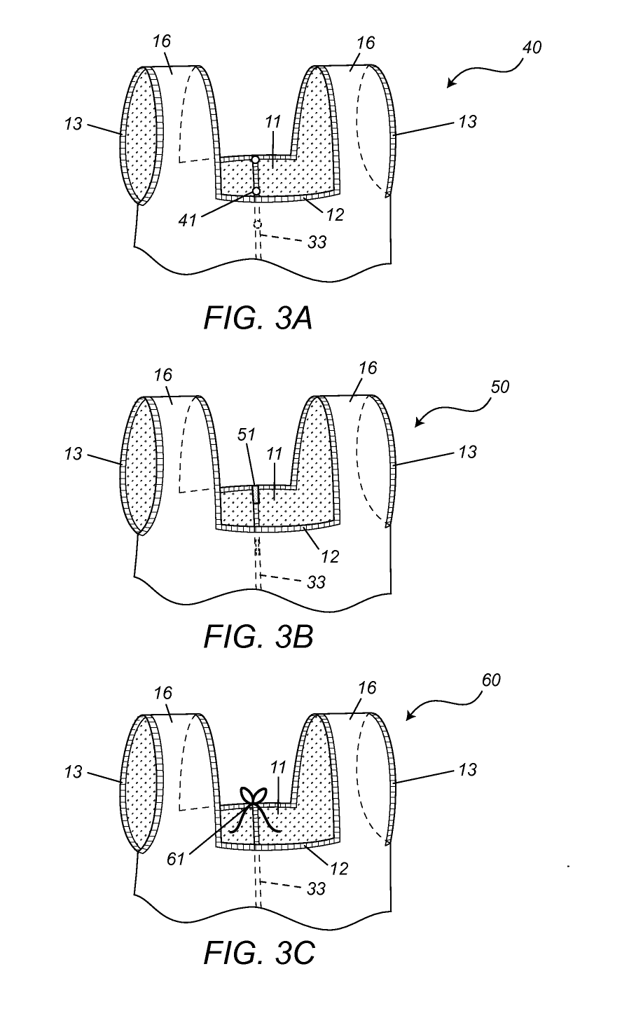

Referring to FIG. 2, the back sheet 31 may include a slit 33 to allow for ease in fitting the degradable emergency diaper liner over the wearer's clothing. In some embodiments, a back portion closure comprising a slit 33 longitudinally from the neck portion to between the mid-back and the waist of the wearer. In some embodiments, the degradable emergency diaper liner comprises a back portion closure comprising at least one back portion fastener 32 selected from at least one pair of drawstrings, at least one snap or at least one button. In some embodiments, the drawstrings are tightened by pulling on the drawstrings and fastened with a knot, at least one snap or at least one button or at least one Velcro™ tab. As illustrated with FIGS. 3A, 3B and 3B, the back portion fastener 32, may comprise at least one snap or at least one button 41, at least one Velcro™ tab 51, drawstrings 61, or combinations thereof.

Referring to FIG. 1, front sheet 17 may be a continuous front side sheet. Moreover, with reference to FIGS. 1 and 2, the leg portions 18, comprise at least one leg portion fastener selected from at least one pair of drawstrings 15, at least one snap or at least one button or at least one Velcro™ tab. In some embodiments, that terminal ends of the leg portions comprise a closure capable of tightening the leg apertures. In some embodiments, the leg portion closures are tightened with at least one fastener selected from at least one pair of drawstrings, at least one snap or button. In some embodiments, the terminal ends of the leg portions comprise a hem 14 having a drawstring disposed therein. Together, the leg portion drawstrings 15 and the hem 14 comprise a leg aperture. Tightening the leg apertures functions to hold the diaper against the legs of the wearer in order to provide a seal which prevents any leakage. The at least one leg portion drawstrings 15 effectively contracts the cuff material adjacent to it around the legs of the wearer. In some embodiments, the leg portions are of a length between about 2-8 inches from the inseam 20 to the terminal end of the leg portions.

The degradable emergency diaper liner may be constructed with a joining means like hot melt adhesives or any other similar means. The longitudinal side edges 19, shoulder portions 16, front sheet 17, back sheet 31, leg portions 18 make up the degradable emergency diaper liner periphery.

In some embodiments, the inner portion 11 of the degradable emergency diaper liner 30 as depicted with FIG. 2 comprises a mixture of a cellulose and with a superabsorbent polymer. The main functions of the SAP component are the liquid absorption and retention in order to avoid any leakage. The mixture of SAP/cellulose may vary in component proportions. In some embodiments, the inner portion 11 is coated with baby powder.

The degradable emergency diaper liner may be manufactured from any flexible and degradable plastic which is impervious to liquid. The components used to prepare the plastic formulation may be biologically-derived. In some embodiments, the liner is constructed from biodegradable plastic. In some embodiments, the liner is constructed from a compostable plastic. In some embodiments, the biodegradable plastic is derived from polybutylene adipate co-terephthalate (PBAT), thermoplastic starch, cellulose esters polyhydroxyalkanoates (PHAs), polybutylene adipate co-succinate co-terephthalate (PBAST), polyvinyl alcohol (PVOH), polybutylene succinate (PBS), poly-3-hydroxybutyrate (PHB), polyhydroxyvalerate (PHV) and polyhydroxyhexanoate (PHH), polylactic acid (PLA), polyglycolic acid, polyesters, polyurethanes, Polyamide 11, Polyamide 410 or combinations thereof. In some embodiments, the liner is derived from polyethylene, polypropylene or a starch blend thereof.

In some embodiments, the liner is constructed from an oxo-biodegradable plastic derived from polyethylene and/or polypropylene and a prodegradant. It should be appreciated that any part of the diaper containing a polyolefin may be made degradable such as by adding a prodegradant to the polyolefin. The polyolefin may be combined with the prodegradant prior to being mixed with the primary to aid in the incorporation of the prodegradant into the polyolefin. Additional examples of polyolefins include low density polyethylene (LDPE), linear low density polyethylene (LLDPE), polypropylene, polybutylene and copolymers of ethylene such as polyethylene-vinyl acetate (EVA), polyethylene-acrylic acid (EAA), polyethylene-methacrylic acid EMA) or copolymers of ethylene or propylene with the lower olefins such as, butene-1, pentene-1, hexene or octene.

The prodegradant is a metal compound selected from the group consisting of cobalt, cerium, and iron. It is understood that the metal may be in ionic form. Other suitable metals are aluminum, antimony, barium, bismuth, cadmium, chromium, copper, gallium, lanthanum, lead, lithium, magnesium, mercury, molybdenum, nickel, potassium, rare earths, silver, sodium, strontium, tin, tungsten, vanadium, yttrium, zinc or zirconium.

The metal compound may be in any suitable form for incorporation into a polyolefin material. In particular, the metal compound is preferably a metal carboxylate. For example, the metal carboxylate may be a metal stearate or a metal neodeconate. The metal is preferably selected from among the metals described above. Thus, exemplary preferred metal compounds include, but are not limited to, cobalt stearate, iron stearate, and cerium stearate. The metal compound is most preferably cobalt stearate.

In some embodiments, the prodegradant could be substituted by any other prodegradant that produces the same degradability effect and characteristics. The preferred total amount of prodegradant is the one that imparts the degradability characteristics without affecting the diaper performance when used, while allowing a suitable shelf life.

The present disclosure related to a fast, easy and convenient way to quickly protect automobile interiors, furniture, etc. in the event an infant, child or an adult has a urination and/or bowel movement accident while not wearing a diaper or, alternatively if the diaper leaks. The present disclosure relates to a degradable emergency diaper liner for on the go containment of leakage of urine and/or feces through clothing after an accident. The liquid impermeable diaper liner designed to fit over the clothing in order to quickly protect valuable car seats and furniture on the go. Moreover, the degradable emergency diaper liner is designed to be biodegradable and/or compostable to limit the environmental impact of municipal landfills.

The presently disclosed diaper liners are not to be limited in scope by the specific embodiments described herein, which are intended as single illustrations of individual aspects of the presently disclosed methods, and functionally equivalent methods and components are within the scope of the presently disclosed diaper liner. Indeed, various modifications of the presently disclosed diaper liner, in addition to those shown and described herein will become apparent to those skilled in the art from the foregoing description and accompanying drawings. Such modifications are intended to fall within the scope of the appended claims.

What is claimed is:

1. A degradable emergency diaper liner comprising:
an inner and an outer portion;
a front portion;
a back portion;
a bottom portion comprising leg portions; and
an open neck portion comprising shoulder straps;
wherein the front portion extending from a terminal end of the leg portions and the open neck portion and the back portion extending between a terminal end of the leg portions and the open neck portion,
wherein the leg portions comprise leg apertures configured for receiving legs of a wearer, and
wherein each of the shoulder straps are coupled to the front portion and back portion adjacent to the open neck portion for receiving a head and arms of a wearer.

2. The degradable emergency diaper liner of claim 1, wherein the liner is sized to fit over the wearer's clothing in the event of a bowl movement and/or urination.

3. The degradable emergency diaper liner of claim 1, wherein the liner is liquid impermeable.

4. The degradable emergency diaper liner of claim 1, wherein the liner is constructed from a biodegradable plastic.

5. The degradable emergency diaper liner of claim 4, wherein the biodegradable plastic is derived from polybutylene adipate co-terephthalate (PBAT), thermoplastic starch, cellulose esters polyhydroxyalkanoates (PHAs), polybutylene adipate co-succinate co-terephthalate (PBAST), polyvinyl alcohol (PVOH), polybutylene succinate (PBS), poly-3-hydroxybutyrate (PHB), polyhydroxyvalerate (PHV) and polyhydroxyhexanoate (PHH), polylactic acid (PLA), polyglycolic acid, polyesters, polyurethanes, Polyamide 11, Polyamide 410 or combinations thereof.

6. The degradable emergency diaper liner of claim 1, wherein the liner is derived from polyethylene, polypropylene or a starch blend thereof.

7. The degradable emergency diaper liner of claim 1, wherein the liner is constructed from an oxo-biodegradable plastic derived from polyethylene and/or polypropylene and a prodegradant.

8. The degradable emergency diaper liner of claim 1, wherein the liner is constructed from a compostable plastic.

9. The degradable emergency diaper liner of claim 1, wherein the inner portion is coated with baby powder.

10. The degradable emergency diaper liner of claim 1, further comprising a back portion closure comprising a slit longitudinally from the neck portion to between the mid-back and the waist of the wearer.

11. The degradable emergency diaper liner of claim 1, further comprising a back portion closure comprising at least one fastener selected from at least one pair of drawstrings, at least one snap or at least one button.

12. The degradable emergency diaper liner of claim 11, wherein the drawstrings are tightened by pulling on the drawstrings and fastened with a knot, at least one snap or at least one button.

13. The degradable emergency diaper liner of claim 1, further comprising that terminal ends of the leg portions comprise a closure capable of tightening the leg apertures.

14. The degradable emergency diaper liner of claim 13, wherein the leg portion closures are tightened with at least one fastener selected from at least one pair of drawstrings, at least one snap or button.

15. The degradable emergency diaper liner of claim 1, wherein the terminal ends of the leg portions and neck portion comprise a hem having a drawstring disposed therein.

16. The degradable emergency diaper liner of claim 1, wherein the neck portion comprises a hem having a drawstring disposed therein.

17. The degradable emergency diaper liner of claim 1, wherein terminal ends of the shoulder portions comprise a hem.

18. The degradable emergency diaper liner of claim 1, wherein the neck portion fits between the waist and the neckline of the wearer.

19. The degradable emergency diaper liner of claim 1, wherein the leg portions are of a length between about 2-8 inches from the inseam to the terminal end of the leg portions.
